(19) United States
(12) Patent Application Publication
(10) Pub. No.: US 2025/0163497 A1
(43) Pub. Date: May 22, 2025

(54) QUANTIFICATION OF ON-TARGET AND OFF-TARGET GENE EDITING ACTIVITY

(71) Applicant: Drexel University, Philadelphia, PA (US)

(72) Inventors: Ming Xiao, Huntingdon Valley, PA (US); Lahari Uppuluri, Philadelphia, PA (US); Yilin Wang, Philadelphia, PA (US)

(21) Appl. No.: 18/955,225

(22) Filed: Nov. 21, 2024

Related U.S. Application Data
(60) Provisional application No. 63/601,606, filed on Nov. 21, 2023.

(51) Int. Cl. C12Q 1/6809; C12N 9/22; C12N 15/11; G01N 21/64

(52) U.S. Cl. CPC C12Q 1/6809; C12N 9/22; C12N 15/111; G01N 21/6428; C12N 2310/20; G01N 2021/6441

(57) ABSTRACT

Described herein are methods useful for quantification of on-target and off-target gene editing activity. The method comprises establishing a first consensus labeling in a genomic DNA in a first plurality of cells, which comprises introducing a first tag at a first plurality of pre-determined locations in the genomic DNA; performing the genomic editing assay with a nucleotide comprising a second tag; identifying locations of genome editing activities by detecting signals of the second tag in reference to signals of the first tag in the first consensus labeling; and calculating genome editing efficiencies at an on-target location or an off-target location.

### CROSS-REFERENCE TO RELATED APPLICATIONS

This application claims priority under 35 U.S.C. § 119 (e) to U.S. Provisional Patent Application No. 63/601,606, filed Nov. 21, 2023, which is incorporated herein by reference in its entirety.

### BACKGROUND

For on-target analysis in genome editing, targeted sequencing is the default approach. This typically involved a complicated PCR step to amplify the modified genes and then the amplicons are sequenced. This approach is difficult to quantify the on-target editing efficiency and generate an unbiased assessment.

Whole genome sequencing (WGS) is the default approach to an unbiased survey of the full genome for off-target analysis. However, WGS is inefficient since most genomic regions are unmodified and sequencing data is not needed. Moreover, when detection off-target genome modification with low efficiencies, WGS becomes costly and impractical. Thus, WGS is limited by throughput, cost, and efficiency. Other sequencing methods based on target enrichment strategies will produce biased detection and cannot provide true off-target gene editing frequency.

There is a need for method for identifying off-target genome editing and quantifying on-target and off-target editing efficiencies. The present invention addresses this need.

### SUMMARY

In some aspects, the present invention is directed to a method of quantifying on-target or off-target genome editing activities of a genomic editing assay. In certain embodiments, the method comprises forming a map in the genomic DNA in a first plurality of cells. In certain embodiments, forming the map comprises introducing a first tag at a first plurality of pre- determined locations in a genomic DNA. In certain embodiments, the method comprises performing the genomic editing assay with a nucleotide comprising a second tag. In certain embodiments, the method comprises identifying locations of genome editing activities by detecting signals of the second tag in reference to signals of the first tag forming the map. In certain embodiments, the method comprises calculating genome editing efficiencies at an on- target location or an off-target location.

The method allows the detection and quantification of genome editing efficiencies in all regions across the genomic DNA. Specifically, the first tag incorporated into the genomic DNA forms a labeling pattern. The second tag is incorporated in the genomic DNA where genome editing has taken place. As such, the distances between a given second tag introduced by the genome editing and its neighboring first tags provide location information of that genome editing.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrate some aspects of CRISPR-Cas9-based genome editing in accordance with some embodiments. The CRISPR-Cas9 genome-editing system can introduce both on-target gene editing based on gRNA targeting, and off-target gene editing.

FIGS. 2A-2C illustrate certain aspects of a non-limiting genome labeling method in accordance with some embodiments. FIG. 2A: Optical mapping of genomic DNA. A First fluorescent tag is introduced into the genomic DNA at known locations by motif labeling. FIG. 2B: Genome editing. The genome editing step is performed with a second fluorescent tag-labeled nucleotide, which incorporates into the genomic DNA where genome editing takes place. FIG. 2C: Analysis of edited genomes. The signals of the first fluorescent tag appear at known locations in the genomic DNA, thereby providing an optical map. The signals of the second fluorescent tag can appear at any locations where genome editing take place, regardless of whether such editing is caused by desired editing or off-target editing.

FIG. 3 illustrates certain aspects of a de novo assemble of consensus labels (or motif labels) and consensus contigs, in accordance with some embodiments. In FIG. 3, “Reference Hg38” is a reference with consensus labels predicted based on the sequence of Hg38 reference human genome. The DNA molecules are optically analyzed to detect the fluorescence signals of the consensus labels, as well as the fluorescence signals introduced by the genome editing. The analyzed data for individual DNA molecules are then assembled to form the de novo assembly of contigs.

FIG. 4 illustrate certain aspects of the identification of genome editing sites, in accordance with some embodiments. The relative distances between genome editing signals and the nearest consensus label signals are calculated, thereby mapping the genome editing locations to the consensus contigs.

FIGS. 5A-5B illustrate certain aspects of establishing a consensus of genome editing signal locations, in accordance with some embodiments.

FIGS. 6A-6B illustrate certain aspects of a genome editing efficiency determination, in accordance with some embodiments. FIG. 6A: Determination of genome regions with genome editing signals. FIG. 6B: Calculation of the genome editing efficiency.

FIG. 7 demonstrate that the method herein is able to detect targeted genome editing at multiple genomic sites with multiple gRNA molecules, in accordance with some embodiments.

FIGS. 8A-8D demonstrate that the method herein is able to detect genome editing at various modification efficiencies. Genomic DNA was labeled by motif labeling (first fluorescent tag, blue) and edited with CRISPR-Cas9 system to incorporate a ˜10 kbp insert in the presence of nucleotides labeled with second fluorescent tag (red). FIG. 8A: about 50% modification efficiency. FIG. 8B: about 25% modification efficiency. FIGS. 8C-8D: about 5% modification efficiency.

FIGS. 9A-9B demonstrate that the method herein is able to detect genome editing when the insert size is small, in accordance with some embodiments. The method was able to detect a 1.3 kbp insert.

FIG. 10 demonstrates that the method herein is able to detect off-target genome editing, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Traditionally, off-target editing in genome editing, such as CRISPR-Cas9 editing is identified by whole genome sequencing (WGS). However, various shortcomings exist in using WGS for this purpose: firstly, WGS is a resource consuming method; secondly, WGS is inefficient since most genomic regions are unmodified and sequencing data is not needed; in addition, in the case where the percentage of off-target editing is low, many copies of genomic DNA have to be sequences to identify such off-target editing.

In the present invention, an optical-based method to identify off-target editing in genome editing was developed. According to the method, genomic DNA are motif-labeled with a first fluorescent tag at known locations to create an optical map, and edited with nucleotides including a second fluorescent tag. The genomic DNA is then analyzed by an optical method, which identifies the locations of genome editing by locating the signals of the second fluorescent tag in reference to the signals of the first fluorescent tag that forms the optical map. In certain embodiments, this method can quantify 1% of gene modification events for both on-target and off-target genome editing.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, selected materials and methods are described herein. In describing and claiming the present invention, the following terminology will be used.

It is also to be understood that the terminology used herein is for the purpose of describing particular embodiments only, and is not intended to be limiting. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, the term “complementary” generally refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-paring with nucleotides within the complementary region of the second polynucleotide. A first polynucleotide is not completely complementary (i.e., partially complementary) to the second polynucleotide if at least one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands. This is of particular importance in amplification reactions, which depend upon binding between polynucleotide strands. It is well-known in the art that sequences need not be completely complementary in order for hybridization to occur.

An oligonucleotide primer is “complementary” to a target polynucleotide if at least 50% (preferably, 60%, more preferably 70%, 80%, still more preferably 90% or more) nucleotides of the primer form base-pairs with nucleotides on the target polynucleotide.

As used herein, “hybridization” means the pairing of complementary oligomeric compounds (e.g., a single strand of a polynucleotide pairing with an anti-parallel polynucleotide strand). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site.

As used herein, the terms “adapter” and “adaptor” are used interchangeably and refer to a nucleic acid molecule that can be used for manipulation of another nucleic acid molecule. In some aspects, an adaptor comprises at least a portion of at least one barcode. In some aspects, an adaptor comprises at least one barcode. In some aspects, adapters are used during assembly of two or more nucleic acid molecules, such as two or more polynucleotide fragments. In some aspects, adaptors are used for amplification of one or more target nucleic acids. In some aspects, adaptors are used in reactions for sequencing. In some aspects, an adaptor comprises, consists of, or consist essentially of at least one priming site. In some aspects, a nucleic acid molecule can be tagged with an adaptor by, e.g., an amplification reaction using a primer comprising the adaptor. In some aspects, the adaptor comprises an unmasking site. In some aspects, the adaptor comprises a cloning site. Further characteristics of adapters are discussed elsewhere herein.

As used herein, the terms “molecular barcode” and “barcode” refer to a nucleic acid sequence, or a combination of nucleic acid sequences, that can act as a ‘key’ to distinguish or separate a plurality of sequences in a sample. For instance, two nucleic acid molecules can each be tagged with a molecular barcode having a unique nucleic acid sequence, such that the two uniquely tagged nucleic acid molecules are distinguishable from one another based on their respective molecular barcodes during nucleic acid sequencing. Moreover, each of two or more different nucleic acid molecules can be tagged with two or more molecular barcodes, wherein the combination of molecular barcodes used to tag each of the two or more different nucleic acid molecules distinguishes the different nucleic acid molecules. In some aspects, at least one molecular barcode is incorporated into the nucleotide sequence of at least one adaptor and/or at least one primer. In some aspects, at least one molecular barcode is used to tag at least one nucleic acid molecule. In some aspects, molecular barcodes are used for amplification of one or more target nucleic acids. In some aspects, the molecular barcodes are used in reactions for sequencing. In some aspects, a molecular barcode comprises, consists of, or consist essentially of at least one priming site.

As used herein, the terms “amplification” or “amplification reaction” refer to a reaction for generating a copy of a particular polynucleotide sequence or increasing the copy number or amount of a particular polynucleotide sequence. For example, polynucleotide amplification may be a process using a polymerase and a pair of oligonucleotide primers for producing any particular polynucleotide sequence, i.e., the whole or a portion of a target polynucleotide sequence, in an amount that is greater than that initially present. Amplification may be accomplished by the in vitro methods of the polymerase chain reaction (PCR). See generally, PCR Technology: Principles and Applications for DNA Amplification (H. A. Erlich, Ed.) Freeman Press, NY, NY (1992); PCR Protocols: A Guide to Methods and Applications (Innis et al., Eds.) Academic Press, San Diego, CA (1990); Mattila et al., Nucleic Acids Res. 19: 4967 (1991); Eckert et al., PCR Methods and Applications 1: 17 (1991); PCR (McPherson et al. Ed.), IRL Press, Oxford; and U.S. Pat. Nos. 4,683,202 and 4,683,195. Other amplification methods include, but are not limited to: (a) ligase chain reaction (LCR) (see Wu and Wallace, Genomics 4: 560 (1989) and Landegren et al., Science 241: 1077 (1988)); (b) transcription amplification (Kwoh et al., Proc. Natl. Acad. Sci. USA 86: 1173 (1989)); (c) self-sustained sequence replication (Guatelli et al., Proc. Natl. Acad. Sci. USA, 87: 1874 (1990)); and (d) nucleic acid based sequence amplification (NABSA) (see, Sooknanan, R. and Malek, L., Bio Technology 13: 563-65 (1995)).

As used herein, the term “amplification reaction conditions” refers to any mixture of reagents that promotes an amplification reaction.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “expression” as used herein is defined as the transcription and/or translation of a particular nucleotide sequence driven by its promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state through the actions of a human being. For example, a nucleic acid or a protein naturally present in a living animal is not “isolated,” but the same nucleic acid or protein partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

The term “recombinant polypeptide” as used herein is defined as a polypeptide produced by using recombinant DNA methods.

The term “recombinant DNA” as used herein is defined as DNA produced by joining pieces of DNA from different sources.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of an immunogenic composition includes, e.g., subcutaneous (s.c.), intravenous (i.v.), intramuscular (i.m.), or intrasternal injection, or infusion techniques.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “substantially the same” amino acid sequence is defined as a sequence with at least 70%, preferably at least about 80%, more preferably at least about 85%, more preferably at least about 90%, even more preferably at least about 95%, and most preferably at least 99% homology with another amino acid sequence, as determined by the FASTA search method in accordance with Pearson & Lipman, 1988, Proc. Natl. Inst. Acad. Sci. USA 85:2444-48.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Method of Analyzing Genome Editing Result

In some aspects, the present invention is directed to a method of analyzing a genomic editing result.

In some embodiments, the method confirms on-target genome editing, and detects and quantifies off-target genome editing.

In some embodiments, the method comprises forming a consensus labeling, such as an optical consensus labeling, in the genomic DNA in a first plurality of cells.

In some embodiments, the consensus labeling is formed by introducing a first tag, such as a first fluorescent tag, at a first plurality of pre-determined locations in a genomic DNA.

In some embodiments, the introduction of the first tag provides signals, such as visual signals, at predetermined locations of the genomic DNA.

In some embodiments, the first tag is introduced with a restriction enzyme cutting, a nicking endonuclease, or a methyltransferase.

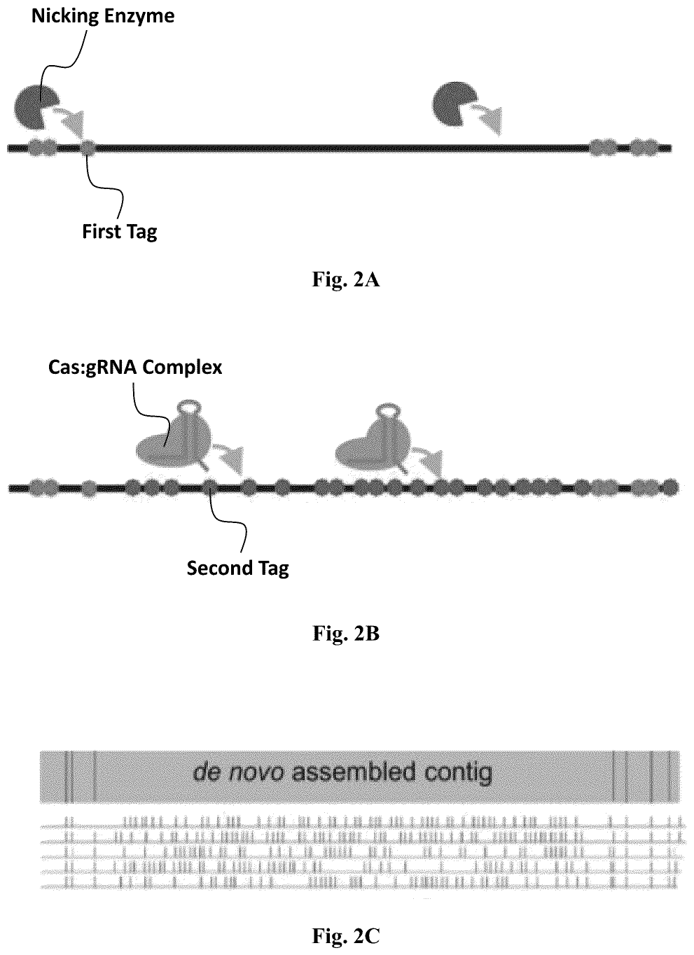

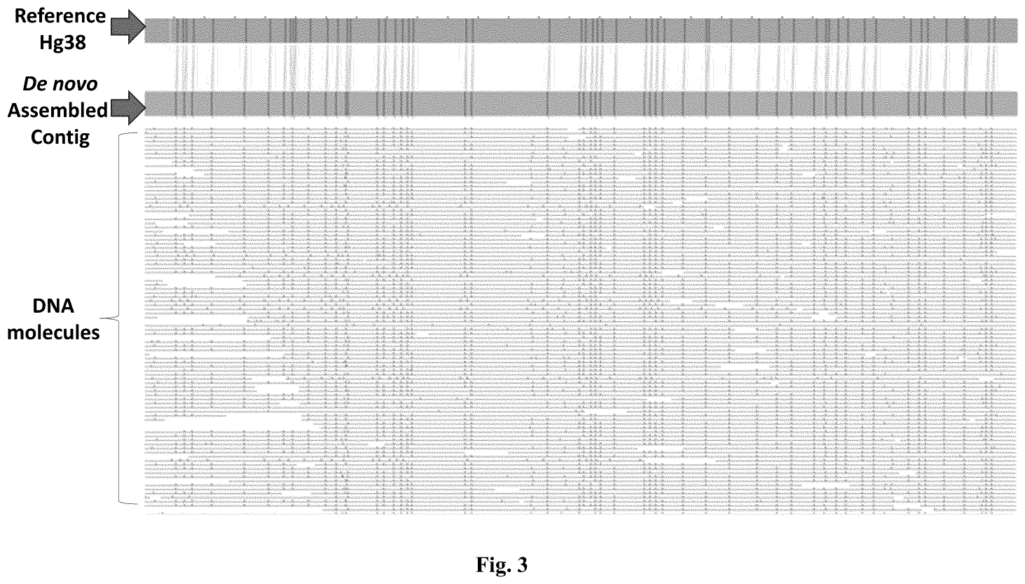

Referring to FIG. 2A, in some embodiments, first tag is introduced with a nicking endonuclease, such as Nt.BspQI. Nt.BspQI recognizes the sequence GCTCTTC, and nicks double-stranded DNA molecules such locations. In human genome, Nt.BspQI nicking sites exist at an average interval of about 8 kb with a known pattern.

In some embodiments, the cells are treated with a tag labeled nucleotide (such as a fluorescent tag labelled nucleotide). Referring to FIG. 2A, in some embodiments, after the nicking endonuclease nicks the DNA at the pre-determined locations, cellular DNA repair machinery repairs the nicks using the fluorescent tag labeled nucleotides, thereby incorporating the second fluorescent tag into the genomic DNA at the predetermined locations. In the case where nicking endonuclease is Nt.BspQI and the genome is a human genome, the fluorescent tag is incorporated at an average interval of about 8 kb with the known pattern.

In some embodiments, the first tag is introduced by a Cas-based nickase editing (such as a Cas9-based nickase editing), which nicks the genomic DNA by targeting a sequence determined by the guide RNA in complex with the Cas nickase. In some embodiments, the first tag is attached to a nucleotide and incorporated into the genomic DNA by cellular DNA repair machinery in response to the nicking by the Cas nickase.

Sometimes, the first tags introduced by one single consensus labeling do not provide a sufficiently detailed map. Accordingly, in some embodiments, the method further comprises establishing an additional consensus labeling by introducing an additional first tag at a second plurality of pre-determined locations in the genomic DNA. For example, the genomic DNA can be treated with both Nt.BspQI targeting GCTCTTC, and a Cas-based nickase targeting a sequence as determined by the complexing gRNA.

Methods of consensus labeling are also described in US 2018/0105867 A1 and Abid et al. (Nucleic Acids Res. 2020 Nov. 24;49 (2): e8), the entireties of which are hereby incorporated herein by reference.

The signals of the first tag (and/or the second tag) can be visualized by various optical method, electric method, and the like. For example, when the tag is a fluorescent tag, its signal can be visualized by any suitable optical methods. The first/second tags can be detected using methods such as nanochannel, nanopore or a nanogap.

In some embodiments, the genomic editing assay is performed with a nucleotide comprising a second tag such that the second tag is incorporated into the genomic DNA in locations where the genomic editing has taken place. In some embodiments, the genome editing assay changes one or more nucleotides or inserts one or more nucleotides in the genomic DNA. In some embodiments, the genome editing assay is a CRISPR-Cas9 based method.

Referring to FIG. 2B, in some embodiments, the Cas:gRNA complex used to perform the genome editing breaks the genomic, which is then repaired by cellular DNA repair mechanism. The second tag is introduced in the form of a nucleotide labeled therewith, and incorporated into the genomic DNA at the editing locations by polymerase when the cells repair the DNA break.

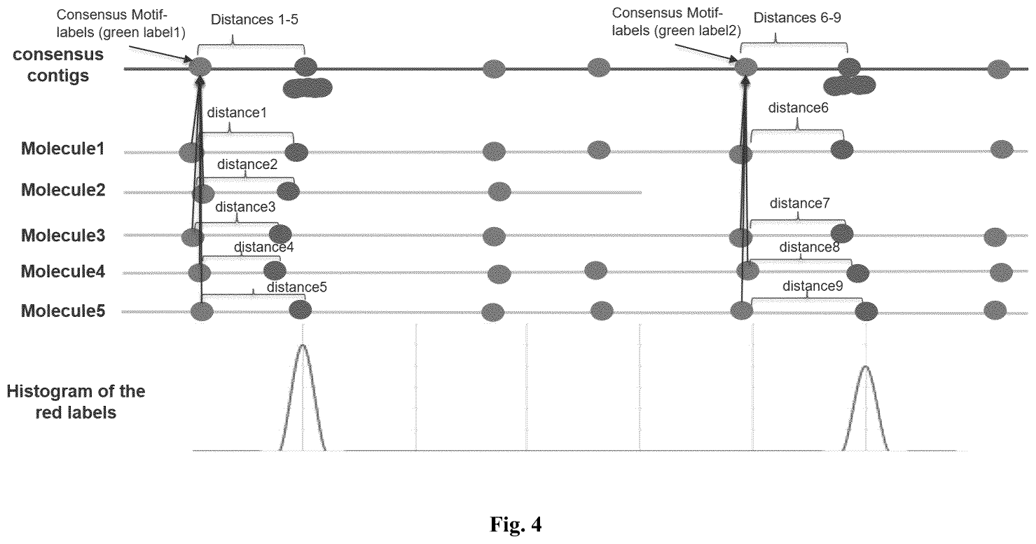

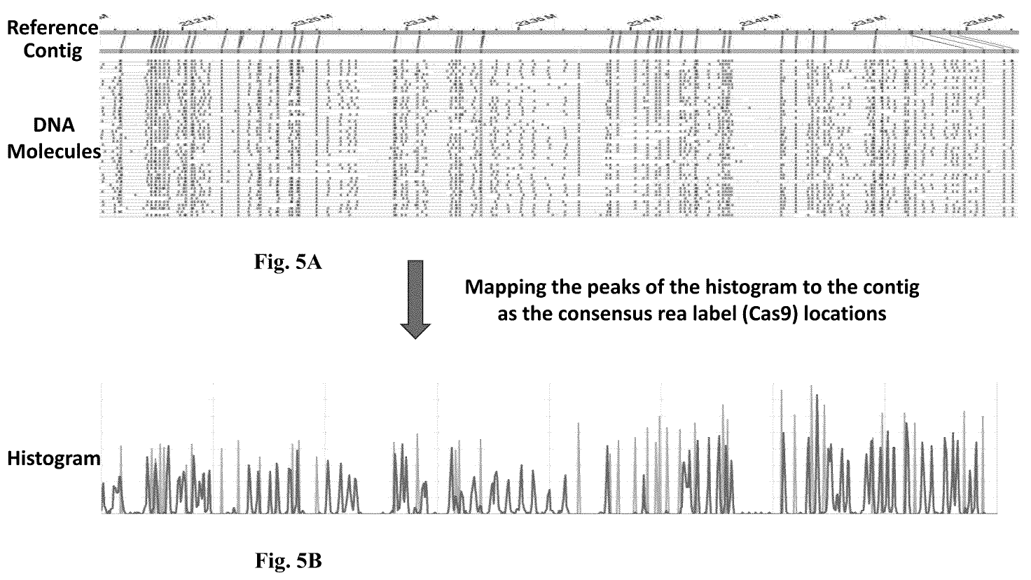

In some embodiments, the method herein further comprises identifying locations of genome editing activities by detecting signals of the second tag in reference to signals of the first and/or second consensus labeling in the genomic DNA. Referring to FIG. 4, in some embodiments, the genome editing locations are determined by calculating the relative distance between second tags in the genomic DNA to their nearest first tags (consensus motif labels) on contigs.

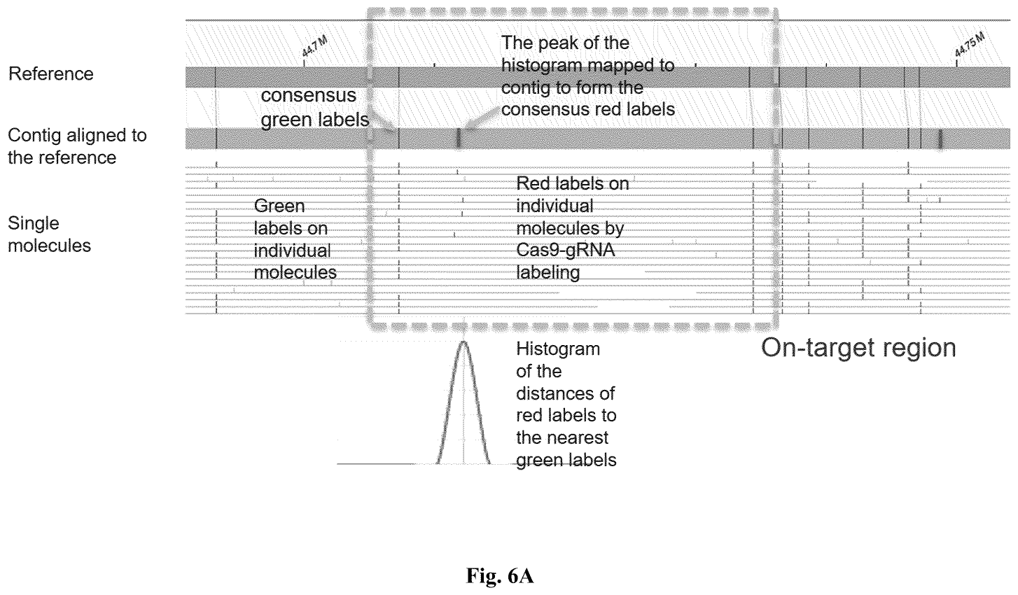

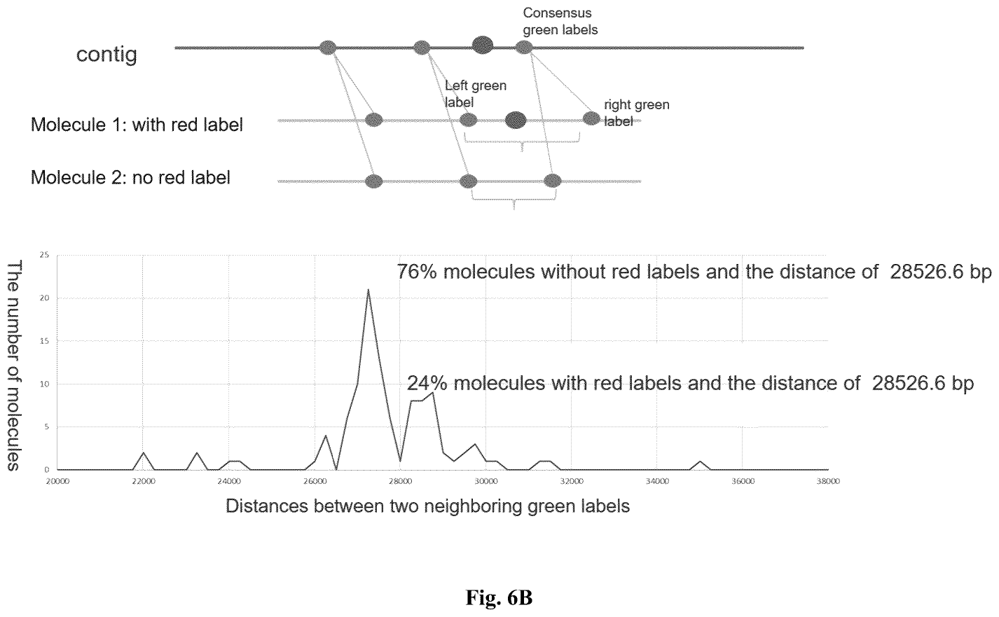

In some embodiments, the method further comprises calculating genome editing efficiencies at an on-target location or an off-target location. Referring to FIGS. 6A-6B, the genome editing efficiencies at a given location is calculated according to a ratio between a number of genomic DNA molecules that contain second tag at this location and a total number of analyzed genomic DNA molecules.

In some embodiments, calculating genome editing efficiencies comprises calculating a ratio between copies of genomic DNA having modification at the on-target location or the off- target location and total copies of the genomic DNA.

In some embodiments, the method herein further comprises determining background genome editing activities of the genome editing assay.

In some embodiments, determining background genome editing activities of the genome editing assay comprises: forming a consensus labeling in the genomic DNA in a second plurality of cells; performing a mock genomic editing assay, wherein the mock genomic editing assay has no specificity toward a sequence in the genomic DNA, but otherwise the same as the genomic editing assay performed on the first plurality of cells; and calculating non-specific genome editing efficiencies along the genomic DNA.

In some embodiments, the genomic editing assay is a CRISPR-Cas9 based genome editing method, and the mock genomic editing assay is performed with a gRNA having no target sequence in the genomic DNA.

### EXAMPLES

The present specification further describes in detail by reference to the following experimental examples. These examples are provided for purposes of illustration only, and are not intended to be limiting unless so specified. Thus, the present specification should in no way be construed as being limited to the following examples, but rather, should be construed to encompass any and all variations which become evident as a result of the teaching provided herein.

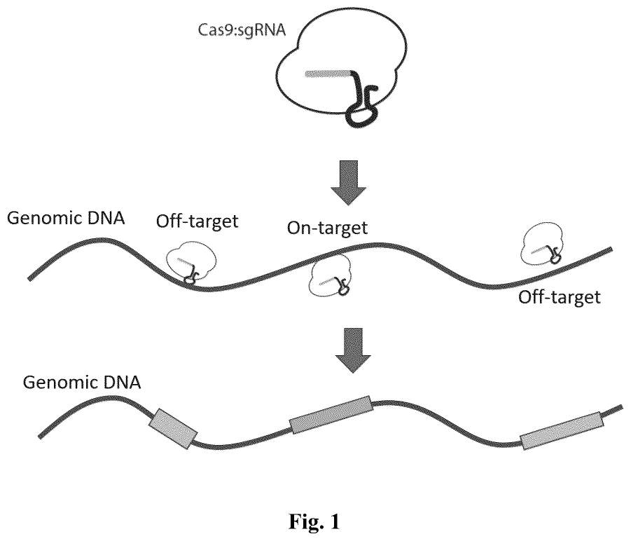

FIG. 1 illustrate some aspects of CRISPR-Cas9-based genome editing in accordance with some embodiments. The CRISPR-Cas9 genome-editing system can introduce both on-target gene editing based on guide RNA (gRNA) targeting, and off-target gene editing.

FIGS. 2A-2C illustrate certain aspects of a non-limiting genome labeling method in accordance with some embodiments. FIG. 2A: Optical mapping of genomic DNA. A First fluorescent tag is introduced into the genomic DNA at known locations by motif labeling. FIG. 2B: Genome editing. The genome editing step is performed with a second fluorescent tag-labeled nucleotide, which incorporates into the genomic DNA where genome editing takes place. FIG. 2C: Analysis of edited genomes. The signals of the first fluorescent tag appear at known locations in the genomic DNA, thereby providing an optical map. The signals of the second fluorescent tag can appear at any locations where genome editing take place, regardless of whether such editing is caused by desired editing or off-target editing.

FIG. 3 illustrates certain aspects of a de novo assemble of consensus labels (or motif labels) and consensus contigs, in accordance with some embodiments. In FIG. 3, “Reference Hg38” is a reference with consensus labels predicted based on the sequence of Hg38 reference human genome. The DNA molecules are optically analyzed to detect the fluorescence signals of the consensus labels, as well as the fluorescence signals introduced by the genome editing. The analyzed data for individual DNA molecules are then assembled to form the de novo assembly of contigs.

FIG. 4 illustrate certain aspects of the identification of genome editing sites, in accordance with some embodiments. The relative distances between genome editing signals and the nearest consensus label signals are calculated, thereby mapping the genome editing locations to the consensus contigs.

FIGS. 5A-5B illustrate certain aspects of establishing a consensus of genome editing signal locations, in accordance with some embodiments.

FIGS. 6A-6B illustrate certain aspects of a genome editing efficiency determination, in accordance with some embodiments. FIG. 6A: Determination of genome regions with genome editing signals. FIG. 6B: Calculation of the genome editing efficiency.

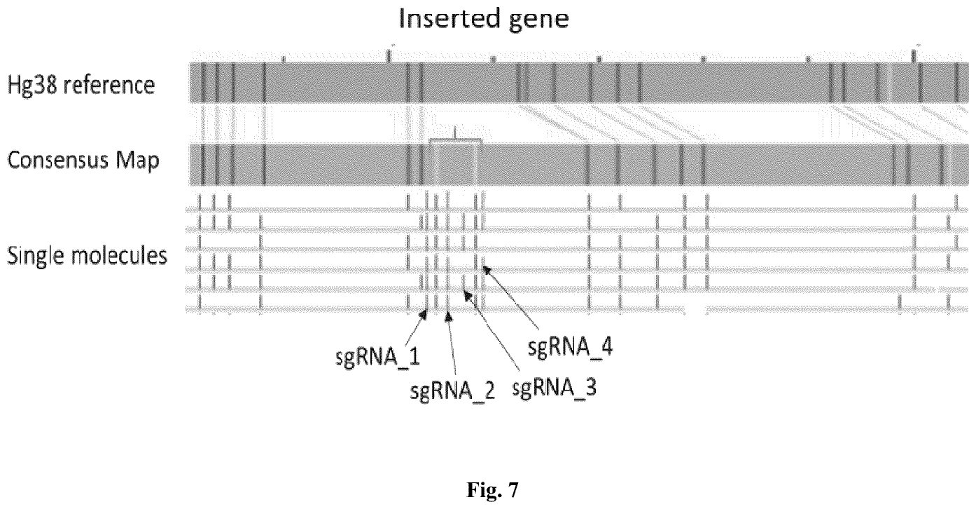

FIG. 7 demonstrate that the method herein is able to detect targeted genome editing at multiple genomic sites with multiple gRNA molecules, in accordance with some embodiments.

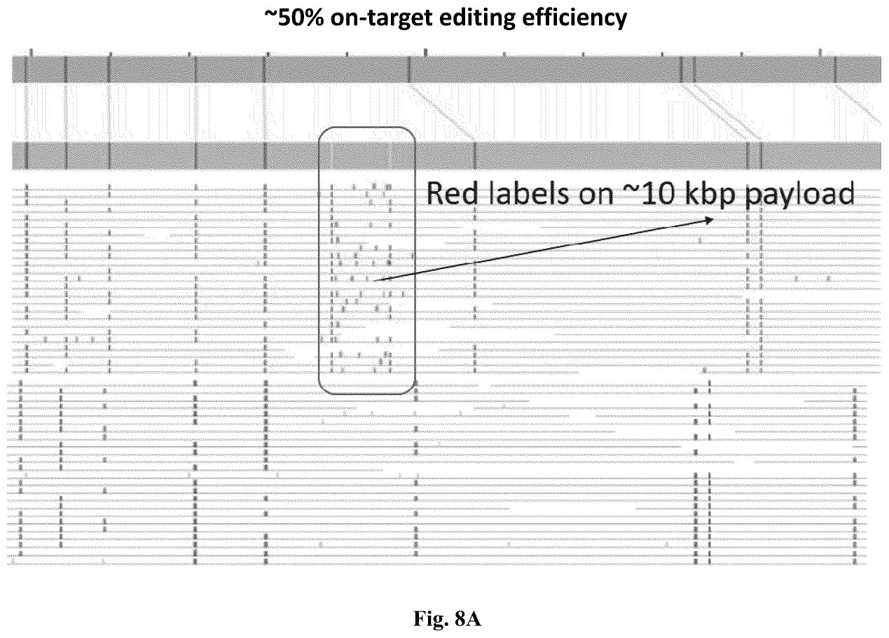

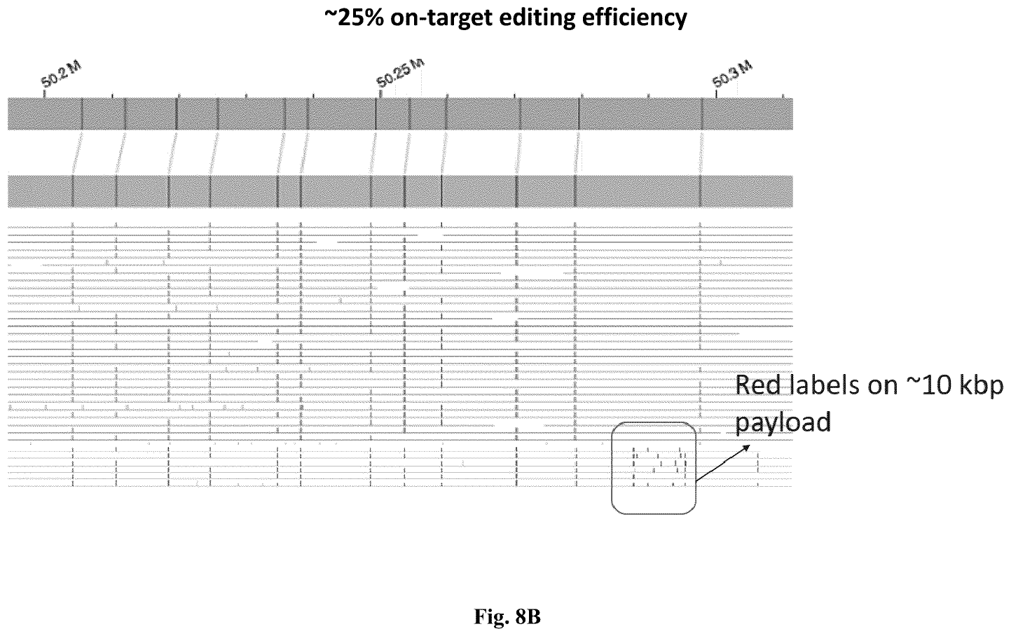

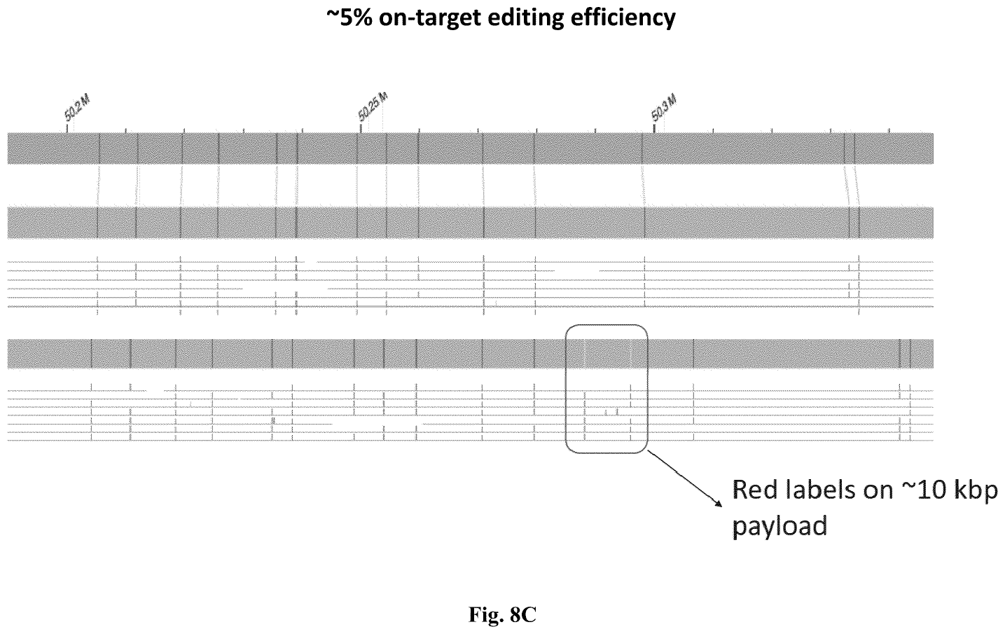

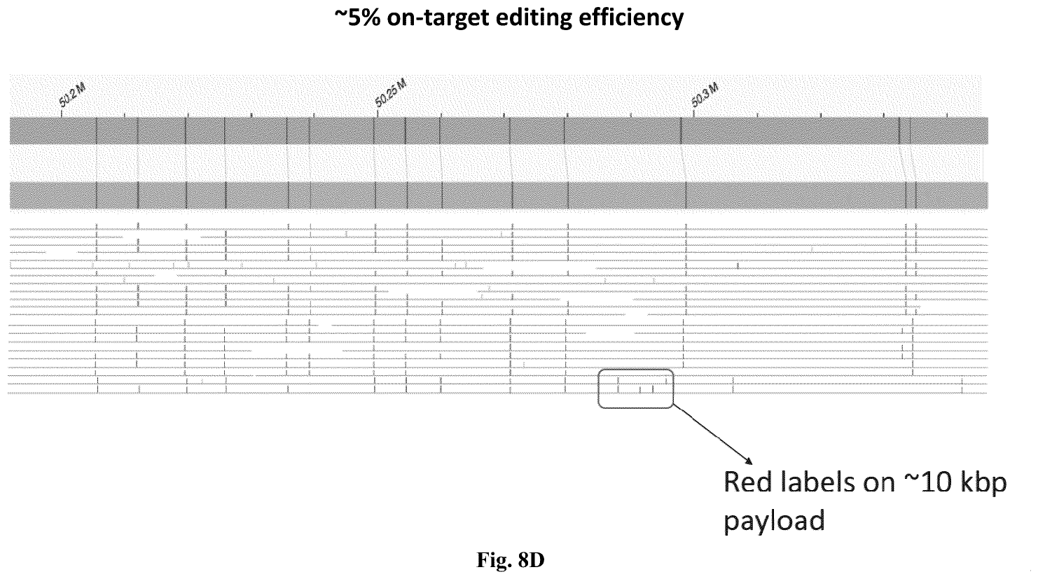

FIGS. 8A-8D demonstrate that the method herein is able to detect genome editing at various modification efficiencies. Genomic DNA was labeled by motif labeling (first fluorescent tag, blue) and edited with CRISPR-Cas9 system to incorporate a ˜10 kbp insert in the presence of nucleotides labeled with second fluorescent tag (red). FIG. 8A: about 50% modification efficiency. FIG. 8B: about 25% modification efficiency. FIGS. 8C-8D: about 5% modification efficiency.

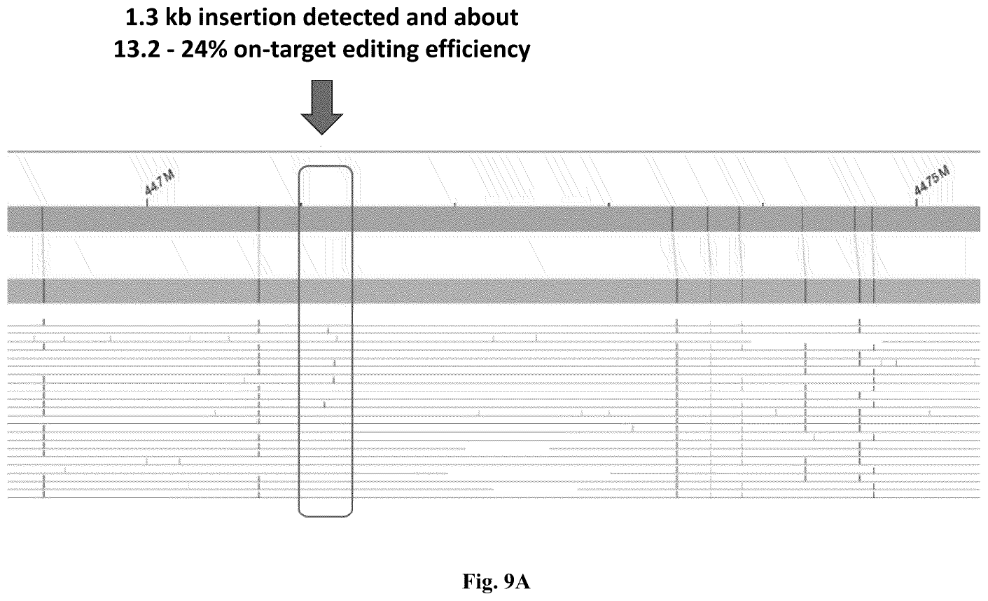

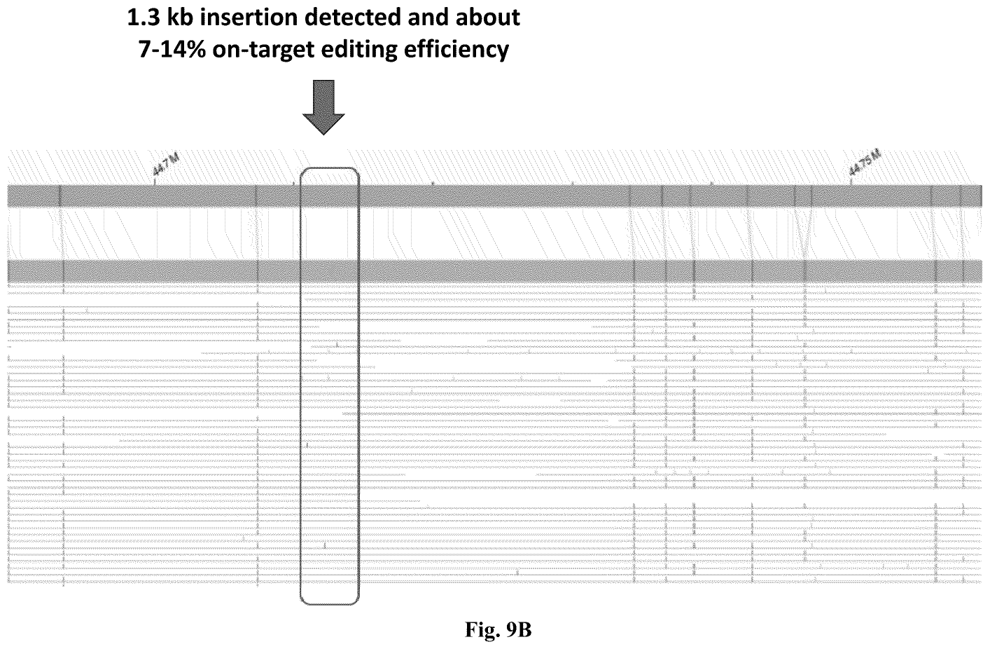

FIGS. 9A-9B demonstrate that the method herein is able to detect genome editing when the insert size is small, in accordance with some embodiments. The method was able to detect a 1.3 kbp insert.

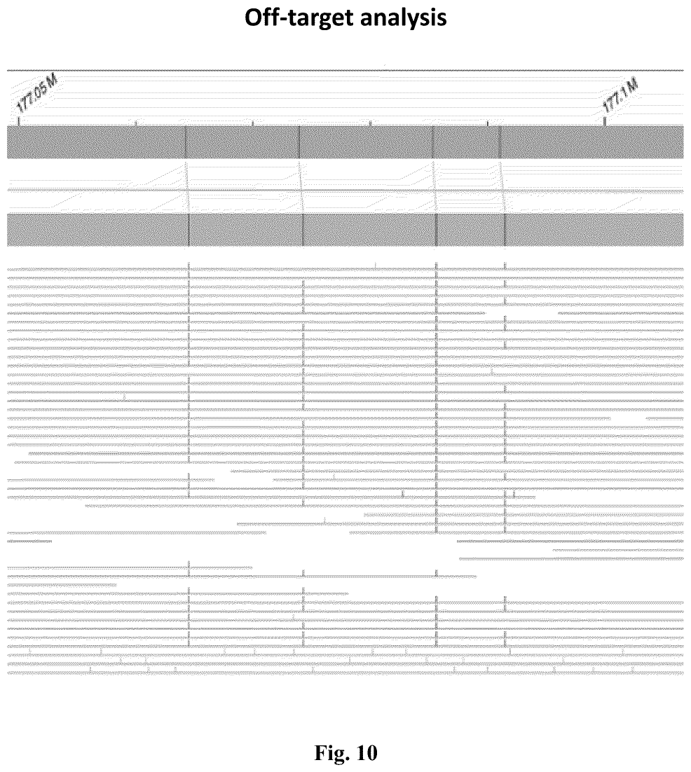

FIG. 10 demonstrates that the method herein is able to detect off-target genome editing, in accordance with some embodiments.

### Enumerated Embodiments

In some aspects, the present invention is directed to the following non-limiting embodiments:

Embodiment 1: A method of quantifying on-target or off-target genome editing activities of a genomic editing assay, the method comprising:
establishing a consensus labeling in a genomic DNA in a first plurality of cells, which comprises introducing a first tag at a first plurality of pre-determined locations in the genomic DNA;
performing the genomic editing assay with a nucleotide comprising a second tag;
identifying locations of genome editing activities by detecting signals of the second tag in reference to signals of the first tag in the consensus labeling; and
calculating genome editing efficiencies at an on-target location or an off-target location.

Embodiment 2: The method of Embodiment 1, wherein the genome editing assay changes one or more nucleotides or inserts one or more nucleotides in the genomic DNA.

Embodiment 3: The method of Embodiment 1 or 1, wherein
(a) the first tag and the second tag each independently capable of providing an optical signal, an electric signal, or
(b) one or both of the first tag and the second tag are fluorescent tags.

Embodiment 4: The method of any one of Embodiments 1-3, wherein the signals of the first tag and the second tag are detected by a nanochannel method, a nanopore method or a nanogap method.

Embodiment 5: The method of any one of Embodiments 1-3, wherein, in establishing the consensus labeling, the first tag is introduced into the first plurality of pre-determined locations as a nucleotide comprising the first tag.

Embodiment 6: The method of any one of Embodiments 1-5, wherein, in establishing the consensus labeling, the first tag is introduced with a restriction enzyme cutting, a nicking endonuclease, a methyltransferase, or a Cas-based nickase: guide RNA (gRNA) complex.

Embodiment 7: The method of any one of Embodiments 1-6, wherein, in establishing the consensus labeling, the first tag is introduced as a nucleotide comprising the first tag in response to a nicking by the nicking endonuclease Nt.BspQI.

Embodiment 8: The method of any one of Embodiments 1-7, wherein the first tag is introduced as a nucleotide comprising the first tag in response to a nicking by a Cas9 nickase: gRNA complex.

Embodiment 9: The method of any one of Embodiments 1-8, wherein performing the genomic editing assay comprises editing the genomic DNA to introduce a strand break in the genomic DNA, and repair the genomic DNA with a nucleotide comprising the second tag.

Embodiment 10: The method of any one of Embodiments 1-9, wherein the genomic editing assay is a CRISPR-based editing assay, optionally a CRISPR-Cas9 editing assay.

Embodiment 11: The method of any one of Embodiments 1-10, wherein identifying locations of genome editing activities comprises measuring a relative distance between a second tag signal and nearest first tag signals thereto.

Embodiment 12: The method of any one of Embodiments 1-11, wherein calculating genome editing efficiencies comprises calculating a ratio between copies of genomic DNA having modification at the on-target location or the off-target location and total copies of the genomic DNA.

Embodiment 13: The method of any one of Embodiments 1-12, wherein genome editing efficiencies is calculated in consideration of background genome editing activities of the genome editing assay, and wherein the method further comprises determining the background genome editing activities.

Embodiment 14: The method of Embodiment 13, wherein determining background genome editing activities of the genome editing assay comprises:
forming a consensus labeling in the genomic DNA in a second plurality of cells;
performing a mock genomic editing assay, wherein the mock genomic editing assay has no specificity toward a sequence in the genomic DNA, but otherwise the same as the genomic editing assay performed on the first plurality of cells; and
calculating non-specific genome editing efficiencies along the genomic DNA.

Embodiment 15: The method of Embodiment 14, wherein the genomic editing assay is a CRISPR based genome editing method, and wherein the mock genomic editing assay is performed with a gRNA having no complimentary sequence in the genomic DNA.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of quantifying on-target or off-target genome editing activities of a genomic editing assay, the method comprising:
establishing a consensus labeling in a genomic DNA in a first plurality of cells, which comprises introducing a first tag at a first plurality of pre-determined locations in the genomic DNA;
performing the genomic editing assay with a nucleotide comprising a second tag;
identifying locations of genome editing activities by detecting signals of the second tag in reference to signals of the first tag in the consensus labeling; and
calculating genome editing efficiencies at an on-target location or an off-target location.

2. The method of claim 1, wherein the genome editing assay changes one or more nucleotides or inserts one or more nucleotides in the genomic DNA.

3. The method of claim 1, wherein
(a) the first tag and the second tag each independently capable of providing an optical signal, an electric signal, or
(b) one or both of the first tag and the second tag are fluorescent tags.

4. The method of claim 1, wherein the signals of the first tag or the second tag are detected by a nanochannel method, a nanopore method or a nanogap method.

5. The method of claim 1, wherein, in establishing the consensus labeling, the first tag is introduced into the first plurality of pre-determined locations as a nucleotide comprising the first tag.

6. The method of claim 1, wherein, in establishing the consensus labeling, the first tag is introduced with a restriction enzyme cutting, a nicking endonuclease, a methyltransferase, or a Cas-based nickase: guide RNA (gRNA) complex.

7. The method of claim 1, wherein, in establishing the consensus labeling, the first tag is introduced as a nucleotide comprising the first tag in response to a nicking by the nicking endonuclease Nt.BspQI.

8. The method of claim 1, wherein the first tag is introduced as a nucleotide comprising the first tag in response to a nicking by a Cas9 nickase: gRNA complex.

9. The method of claim 1, wherein performing the genomic editing assay comprises editing the genomic DNA to introduce a strand break in the genomic DNA, and repair the genomic DNA with a nucleotide comprising the second tag.

10. The method of claim 1, wherein the genomic editing assay is a CRISPR-based editing assay, optionally a CRISPR-Cas9 editing assay.

11. The method of claim 1, wherein identifying locations of genome editing activities comprises measuring a relative distance between a second tag signal and nearest first tag signals thereto.

12. The method of claim 1, wherein calculating genome editing efficiencies comprises calculating a ratio between copies of genomic DNA having modification at the on-target location or the off-target location and total copies of the genomic DNA.

13. The method of claim 1, wherein genome editing efficiencies is calculated in consideration of background genome editing activities of the genome editing assay, and wherein the method further comprises determining the background genome editing activities.

14. The method of claim 13, wherein determining background genome editing activities of the genome editing assay comprises:
forming a consensus labeling in the genomic DNA in a second plurality of cells;
performing a mock genomic editing assay, wherein the mock genomic editing assay has no specificity toward a sequence in the genomic DNA, but otherwise the same as the genomic editing assay performed on the first plurality of cells; and
calculating non-specific genome editing efficiencies along the genomic DNA.

15. The method of claim 14, wherein the genomic editing assay is a CRISPR based genome editing method, and wherein the mock genomic editing assay is performed with a gRNA having no complimentary sequence in the genomic DNA.
